(19) United States
(12) Patent Application Publication
(10) Pub. No.: US 2025/0084439 A1
(43) Pub. Date: Mar. 13, 2025

(54) SYSTEMS AND METHODS FOR APPLYING VOLTAGES WITHIN DROPLET-BASED SYSTEMS

(71) Applicant: DropGenie, Inc., Boston, MA (US)

(72) Inventors: Steve Shih, Montréal (CA); Alison Hirukawa, Charlestown, MA (US); Samuel Little, Montréal (CA)

(73) Assignee: DropGenie, Inc., Boston, MA (US)

(21) Appl. No.: 18/566,181

(22) PCT Filed: Jun. 3, 2022
(86) PCT No.: PCT/US2022/032083

§ 371 Date: Dec. 1, 2023

Related U.S. Application Data
(60) Provisional application No. 63/197,202, filed on Jun. 4, 2021.

(51) Int. Cl. C12N 15/87; C12M 1/42; C12M 3/06

(52) U.S. Cl. CPC C12N 15/87; C12M 23/16; C12M 35/02

(57) ABSTRACT

The present disclosure generally relates to systems and methods for applying voltages or currents in droplet-based systems, e.g., to cause electroporation. For example, some embodiments are directed to applying voltage or current to a target droplet via other droplets that physically contact and/or are in ionic communication with that droplet. This may be used, for example, to prevent or reduce contamination from the electrodes applying the voltage. In some cases, the droplets may be present or controlled by a digital microfluidic (DMF) device using pixels within the device. For example, one or more droplets may be defined by pixels within the DMF device, and electrodes for applying such voltages or currents may be present within or near certain pixels. Other embodiments are generally directed to methods for making or using such systems, e.g., to electroporate cells, kits involving such systems, or the like.

### RELATED APPLICATIONS

This application claims the benefit of U.S. Provisional Patent Application Ser. No. 63/197,202, filed Jun. 4, 2021, entitled “Systems and Methods for Applying Voltages within Droplet-Based Systems,” by Shih, et al., incorporated herein by reference in its entirety.

### FIELD

The present disclosure generally relates to systems and methods for applying voltages or currents in droplet-based systems.

### BACKGROUND

Electroporation is a technique where an electric field is applied to cells in order to increase the permeability of the cell membrane, which can be used to transport materials into or out of the cells, for example, DNA for purposes of transfection of the cells. Typically, the cells are suspended in a solution, which is contained within an electroporation cuvette having electrodes on its sides, then relatively high voltages (e.g., thousands of volts) are applied to the electrodes on the sides of the cuvette, causing a voltage drop across the solution which electroporates the cells. However, in the process of creating such high voltages, ions and other chemical by-products are created at the electrodes, which can contaminate or even kill cells that are being electroporated. Accordingly, improvements in electroporation techniques are needed.

### SUMMARY

The present disclosure generally relates to systems and methods for applying voltages or currents in droplet-based systems. The subject matter of the present disclosure involves, in some cases, interrelated products, alternative solutions to a particular problem, and/or a plurality of different uses of one or more systems and/or articles.

One aspect of the present disclosure is generally directed to a digital microfluidic device. In one set of embodiments, the device a plurality of pixels, including a first pixel, a second pixel, and at least one pixel between the first pixel and the second pixel; a first electrode in contact with the first pixel; a second electrode in contact with the second pixel; and a voltage generator able to produce a voltage of at least 10 V between the first electrode and the second electrode.

In another set of embodiments, the device comprises a first electrode, a second electrode separated from the first electrode by at least 10 micrometers, and a voltage generator able to produce a voltage of at least 10 V between the first electrode and the second electrode.

Another aspect of the present disclosure is generally directed to an electroporation system. The system, in some embodiments, comprises a first ion containment system surrounding a first electrode, a second ion containment system surround a second electrode, and a target fluidic droplet in electrical communication with the first electrode and the second electrode. In certain embodiments, when a voltage is applied between the first electrode and the second electrode, ions created at each of the first electrode and the second electrode are contained in the respective first and second ion containment systems.

Still aspect of the present disclosure is generally directed to a method. In one set of embodiments, the method comprises contacting a target fluidic droplet with a first fluidic droplet in ionic communication with a first electrode and a second fluidic droplet in ionic communication with a second electrode, and applying a voltage between the first electrode and the second electrode.

In another set of embodiments, the method comprises applying a voltage of at least 10 V to a target fluidic droplet using a first fluidic droplet and a second fluidic droplet, each in contact with the fluidic droplet.

The method, in yet another set of embodiments, comprises merging a first droplet containing a first fluid with a second droplet containing a second fluid to produce a merged droplet, and applying a voltage to the merged droplet.

In another aspect, the present disclosure encompasses methods of making one or more of the embodiments described herein, for example, devices such as digital microfluidic devices for applying electroporation. In still another aspect, the present disclosure encompasses methods of using one or more of the embodiments described herein, for example, devices such as digital microfluidic devices for applying electroporation.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1-3 illustrate certain embodiments for applying a voltage to a target droplet;

FIGS. 4-6 illustrate various embodiments in which a target droplet is present between two layers;

FIGS. 7-8 illustrate certain embodiments for applying a voltage to a target droplet using a chain of droplets;

FIG. 9 illustrates the electric field distribution in a target droplet adjacent to two other droplets, in accordance with yet another embodiment;

FIGS. 10A-10B illustrate electroporation using three droplets, in one embodiment;

FIGS. 11A-11B illustrate expression in electroporated droplets, in another embodiment;

FIG. 12 illustrates efficiency as a function of voltage, in yet another embodiment;

FIGS. 13A-13B illustrate voltage optimization data, in still another embodiment;

FIG. 14 illustrates payload testing using three droplets, in another embodiment;

FIG. 15 illustrate payload testing using three droplets, in still another embodiment; and

FIGS. 16A-16C illustrates efficiency and viability in cell populations using electroporation in three droplets, in another embodiment.

### DETAILED DESCRIPTION

The present disclosure generally relates to systems and methods for applying voltages or currents in droplet-based systems, e.g., to cause electroporation. For example, some embodiments are directed to applying voltage or current to a target droplet via other droplets that physically contact and/or are in ionic communication with that droplet. This may be used, for example, to prevent or reduce contamination from the electrodes applying the voltage. In some cases, the droplets may be present or controlled by a digital microfluidic (DMF) device using pixels within the device. For example, one or more droplets may be defined by pixels within the DMF device, and electrodes for applying such voltages or currents may be present within or near certain pixels. Other embodiments are generally directed to methods for making or using such systems, e.g., to electroporate cells, kits involving such systems, or the like.

One aspect of the present disclosure is generally directed to devices and methods for inserting molecules such as biomolecules (such as CRISPR-associated ribonucleoproteins, genes such as gRNA or Cas9, or other molecules described herein, etc.) into living cells via electroporation, for example, for gene editing purposes. In some cases, high viabilities and/or high transport efficiencies may be achieved. According to some embodiments, the cells may be isolated from harmful by-products due to electroporation, while the cells are exposed to a relatively homogenous electric field. In addition, in certain embodiments, the cells may be moved into recovery buffer relatively quickly, e.g., after electroporation. In one set of embodiments, lower voltages or varying cell amounts may be used. For instance, in some cases, lower quantities of cells can be used.

In addition, certain aspects as discussed herein are generally directed to systems and methods for applying a voltage or current to a target droplet using other droplets that are in physical contact or in ionic communication with the target droplet. The target droplet may contain cells or other entities, for instance, to which a voltage or current is to be applied. This may be useful, for example, to cause electroporation to occur in cells within the droplet (e.g., to cause material such as DNA to be transported into cells), or for other applications such as those described herein.

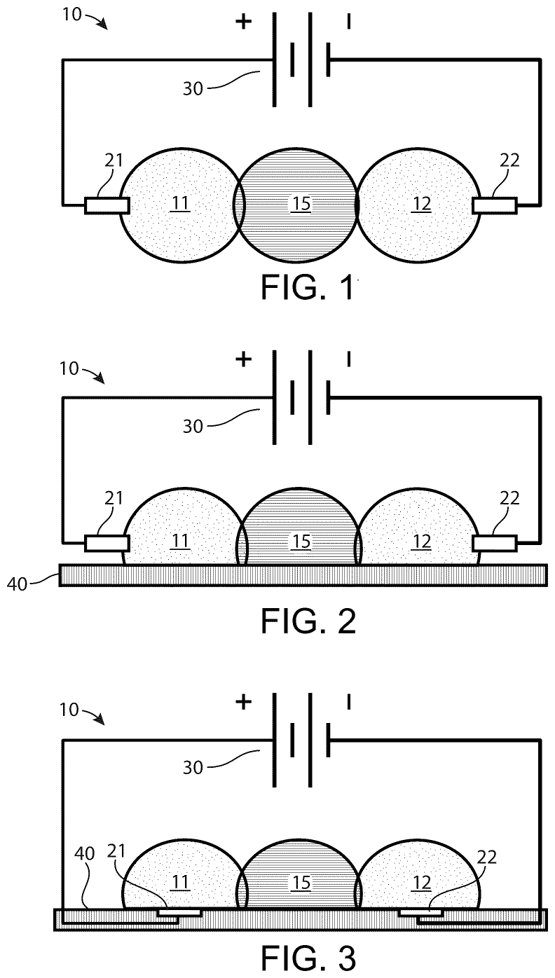

One non-limiting example of such a device is now described with reference to FIG. 1. As will be discussed in more detail below, in other embodiments, other configurations can be used as well. In FIG. 1, device 10 is used to apply a voltage to target droplet 15. A voltage is created using voltage generator 30, which may be, for example, an electrical pulse generator, or another type of generator (e.g., a current generator). The voltage created by voltage generator 30 in this example is then applied via electrodes 21 and 22 to target droplet 15. Electrode 21 is in contact with droplet 11, while electrode 22 is in contact with droplet 12, and each of droplets 11 and 12 are in contact with target droplet 15. In such a system, an ionic communication pathway is formed through which current can flow from electrode 21 to electrode 22 (or vice versa). Accordingly, a voltage (or current) created from voltage generator 30 flows via electrodes 21 and 22 across droplets 11, 12 and 15. It should be understood that within these droplets, the charge carriers that cause current flow are predominantly ions, rather than electrons. Thus, in FIG. 1, target droplet 15 is in ionic communication with electrodes 21 and 22 via droplets 11 and 12, with ions acting as charge carriers flowing through droplets 11 and 12.

However, while FIG. 1 illustrates a situation with a target droplet and two additional droplets that allow the target droplet to be in ionic communication with the electrodes, it should be understood that this is by way of example only, and that in other embodiments, more or fewer droplets and/or electrodes, or other device configurations, can be used. For example, between an electrode and a target droplet may be one, two, three, four, or any other suitable number of droplets. There may also be the same or different numbers of droplets on either side of a target droplet, e.g., to create an ionic communication pathway with an electrode. Examples of these and other embodiments are discussed in more detail herein. In addition, multiple target droplets may be manipulated, e.g., sequentially and/or simultaneously. For example, in certain embodiments, there may be two or more target droplets in communication with a pair of electrodes, and/or with multiple pairs of electrodes. Accordingly, more generally, various aspects of the present disclosure are directed to various systems and methods for applying voltages or currents to a target droplet, and are not necessarily limited to that shown in FIG. 1.

For example, one aspect of the present disclosure is generally directed to systems and methods for applying relatively high voltages to one or more target droplets. The voltages experienced by a target droplet may be, for example, at least 1 V, at least 2 V, at least 3 V, at least 5 V, at least 10 V, at least 20 V, at least 30 V, at least 50 V, at least 100 V, at least 200 V, at least 300 V, at least 500 V, at least 1 kV, etc. In some cases, the voltage may be no more than 1 kV, no more than 500 V, no more than 300 V, no more than 200 V, no more than 100 V, no more than 50 V, no more than 30 V, no more than 20 V, no more than 10 V, no more than 5 V, no more than 3 V, no more than 2 V, no more than 1 V, etc. Combinations of any of these are also possible in certain embodiments, e.g., the voltage experienced by a target droplet may be between 50 V and 200 V, between 500 V and 1 kV, between 10 V and 20 V, etc. The voltages may be created using a using voltage generator or a voltage source. Such voltages may be continuous, and/or applied as electrical pulses, e.g., by using an electrical pulse generator. Those of ordinary skill in the art will be aware of a variety of voltage sources that may be used, some of which are available commercially. In addition, it should be understood that the present disclosure is not limited to only voltage sources, and that in certain embodiments, a current source may be used.

The voltage may be applied to a target droplet for a variety of applications. For example, in one embodiment, the voltage is applied to cause a reaction involving an entity such as a voltage-sensitive compound (e.g., voltage-sensitive dyes such as a quinone, for example, 2,5-dimethyl-1,4-hydroquinone) within the droplet. As another non-limiting example, the application of voltage (or current) to a target droplet may cause the droplet to fuse with other droplets. Without wishing to be bound by any theory, it is believed that voltage or current may cause charges to form on one or more of the droplets, e.g., positive and negative charges or dipoles, which may overcome surface tension and allow some or all of the droplets to fuse together.

As yet another example, in some embodiments, the voltage may be applied to cause electroporation to occur in cells within a target droplet. As is known by those of ordinary skill in the art, electroporation generally involves applying a voltage to cells to cause material to be transported into or out of cells. This can be used, for example, to introduce nucleic acids into cells, e.g., to transfect those cells with the nucleic acids. Non-limiting examples of nucleic acids include DNA or RNA. A variety of genes (for example, contained in plasmids) may accordingly be transfected into cells for various applications such as gene editing, for instance, by using CRISPR genes for gRNA and Cas9, etc. Other examples of genes that can be transfected include, but are not limited to, linear DNA, circular DNA, nucleic acids or fragments that are naturally occurring or are synthesized, etc. In addition, as another non-limiting example, a functional nucleic acid may be transported into a cell; examples include, but are not limited to, Aptamers, nucleic acid enzymes, aptazymes, ribozymes, deoxyribozymes, or the like. Other materials that may be transported into or out of cells include, but are not limited to, proteins, peptides, viruses, hormones, drugs, dyes such as fluorescent dyes, small molecules (e.g., with a molecular weight of less than 1 kDa or less than 2 kDa), charged compounds, chemotherapeutic agents, or the like.

In addition, in some embodiments, more complex pulsing regimes may be applied, e.g., to cells within a target droplet. As a non-limiting example, in one set embodiments, an initial pulse may be applied to cells within a droplet, for instance, to optimize the distribution and alignment of pores in the cell membrane, and then a higher voltage pulse applied, e.g., to cause delivery into the cells. As another non-limiting example, an initial pulse may be applied to cells within a target droplet to distribute the cells in the target droplet, e.g., to facilitate their distribution within the target droplet for a subsequent pulse or electric field that may be applied. Other pulsing regimes are also possible in certain cases.

The target droplets may contain various entities to which a voltage or current is to be applied. For example, according to certain applications, the voltage or current may be applied to cells. Thus, in some embodiments, the target droplet may contain any number of cells and/or cell types. For instance, the cells may be targeted for electroporation, or for other applications. The cells may be adherent, in some embodiments. The cell may be, for example, an isolated cell, a cell aggregate, or a cell found in a cell culture, in a tissue construct containing cells, or the like. Non-limiting examples of cells include immortal cells, primary cells, stem cells, germline cells, zygotes, embryos, or the like. Additional non-limiting examples of cells include, but are not limited to, a microbial cell, e.g., from bacterium or other single-cell organism, a plant cell, or an animal cell. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a mammalian cell, human or non-human mammal, such as a monkey, cow, sheep, goat, horse, rabbit, pig, mouse, rat, dog, or cat. If the cell is from a multicellular organism, the cell may be from any part of the organism. For instance, if the cell is from an animal, the cell may be a cardiac cell, a fibroblast, a keratinocyte, a heptaocyte, a chondracyte, a neural cell, an osteocyte, an osteoblast, a muscle cell, a blood cell, an endothelial cell, an immune cell (e.g., a T-cell, a B-cell, a macrophage, a neutrophil, a basophil, a mast cell, an cosinophil), etc. In one set of embodiments, the cells include mammalian cells. In another set of embodiments, the cells include non-mammalian cells.

The target droplet may contain a single cell type, or more than one cell type, e.g., from the same or different species, from the same or different organisms, etc. In some cases, the target droplet may contain only a single cell. However, in other cases, more than one cell may be present (e.g., which may be the same or different). For instance, a droplet may have at least 5, at least 10, at least 30, at least 50, at least 100, at least 300, at least 500, at least 1,000, at least 3,000, at least 5,000, at least 10,000, at least 30,000, at least 50,000, at least 100,000, at least 300,000, at least 500,000, or at least 1,000,000 cells. In some cases, a droplet may contain no more than 1,000,000, no more than 500,000, no more than 300,000, no more than 100,000, no more than 50,000, no more than 30,000, no more than 10,000, no more than 5,000, no more than 3,000, no more than 1,000, no more than 500, no more than 300, no more than 100, no more than 50, no more than 30, no more than 10, or no more than 5 cells. In addition, in certain embodiments, a droplet may contain a number of cells between any of these ranges. For instance, a target droplet may contain between 50,000 and 100,000 cells, between 5 and 50 cells, between 300 and 1,000 cells, etc.

Electroporation is traditionally performed using electroporation cuvettes, as discussed above. However, due to the size of such cuvettes (typically having a width of 12.5 mm), relatively large numbers of cells and relatively large volume sizes (e.g., hundreds of microliters) are typically required. In contrast, as discussed herein, in some embodiments, smaller numbers of cells, and/or smaller volumes, can be used.

As mentioned, voltage or current can be applied to a target droplet, in accordance with certain embodiments. The target droplet may be, for example, the target to which a voltage is to be applied, for example, a droplet containing one or more cells (for example, to cause electroporation in at least some of the cells, to cause droplet fusion to occur, or the like). In addition, in some cases, more than one of the effects may occur, e.g., sequentially or simultaneously. For example, a target droplet may be fused with other droplets, then electroporated.

In some cases, e.g., when multiple droplets are present between the electrodes, including a target droplet, the droplets may be configured such that a target droplet experiences the largest voltage drop out of all of the droplets forming an ionic communication pathway between the electrodes. In some embodiments, there is a single target droplet in a pathway, although in certain embodiments, 2, 3, 4, or more target droplets may be present.

The target droplet may be in physical contact with an electrode, or there may be one or more other droplets forming an ionic communication pathway connecting the target droplet to the electrodes. The pathway may be such that ions can flow from one electrode to the other, passing through a plurality of droplets, including target droplets. Each of the droplets may be in physical contact with a neighboring droplet, or at least positioned to be in ionic communication with neighboring droplets, e.g., such that there is an ionic communication pathway from one electrode to the other.

The ionic communication pathway may pass between any number of droplets, including target droplets and other droplets. Thus, there may be any number of droplets between a target droplet and an electrode, for example, creating a chain of droplets, and the droplets may be symmetrically or asymmetrically arranged. For example, there may be 1, 2, 3, 4, 5, 6, 7, 8, 9, 10, or any other number of droplets between a target droplet and one electrode, and 1, 2, 3, 4, 5, 6, 7, 8, 9, 10, or any other number of droplets between the target droplet and another electrode. In addition, the number of droplets may be the same or different. The droplets may be linearly arranged between the electrodes, or in some cases, the droplets may be arranged in a non-linear fashion. In addition, in some cases, there may be more than one ionic communication pathway between the electrodes, e.g., passing through different droplets. In some embodiments, there may also be more than two electrodes, e.g., in ionic communication with a target droplet, directly or via one or more additional droplets, etc.

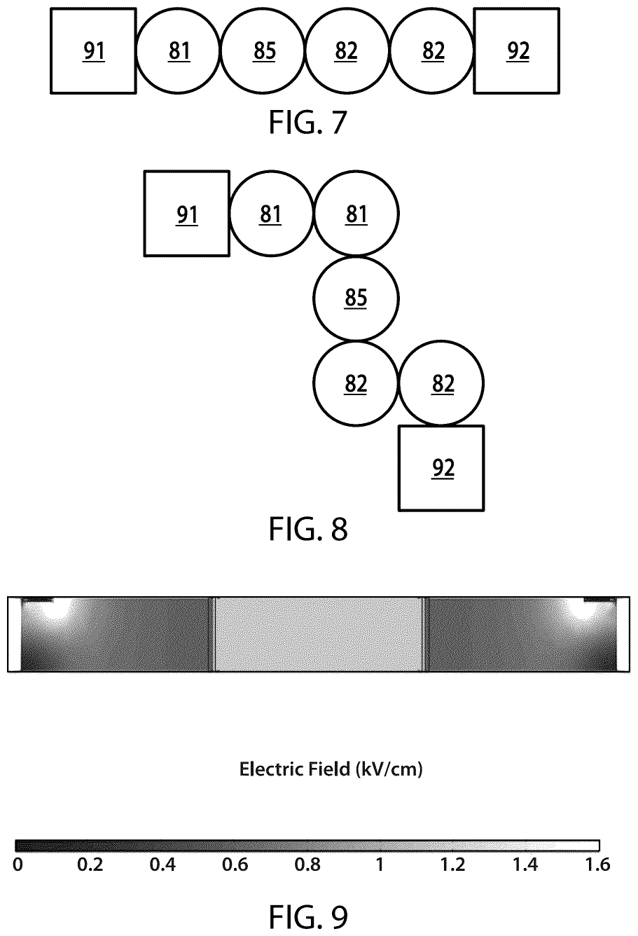

As a non-limiting illustrative example, FIGS. 7 and 8 show (top view) various droplets 81. 82 between electrodes 91, 92 and target droplet 85. These droplets may be linearly arranged or not linearly arranged, depending on the embodiment. For example, in FIG. 7, the droplets are present in a linear pathway between electrodes 91 and 92, while in FIG. 8, the droplets are present in a nonlinear pathway between electrodes 91 and 92. In addition, it should be noted that these figures illustrate that there may be one or more than one droplet between target droplet 85 and electrodes 91 and 92. Configurations such as these may be achieved using a variety of techniques. For example, in some cases, the droplets may be manually positioned between the electrodes. In addition, in certain embodiments, a digital microfluidic device may be used to position droplets between electrodes. Digital microfluidic devices are discussed in more detail below.

The droplets (including target and other droplets) may be of any shape or size, and may each independently be the same or different sizes. For instance, in various embodiments, a droplet may have an average or characteristic diameter of less than 1 cm, less than 5 mm, less than 3 mm, less than 2 mm, less than 1 mm, less than 500 micrometers, less than 300 micrometers, less than 200 micrometers, less than 100 micrometers, less than 75 micrometers, less than 50 micrometers, less than 40 micrometers less than 30 micrometers, less than 25 micrometers, less than 20 micrometers, less than 15 micrometers, less than 10 micrometers, less than 5 micrometers, less than 3 micrometers, less than 2 micrometers, less than 1 micrometer, etc. The average or characteristic diameter of a droplet may also be at least 1 micrometer, at least 2 micrometers, at least 3 micrometers, at least 5 micrometers, at least 10 micrometers, at least 15 micrometers, at least 20 micrometers, at least 25 micrometers, at least 30 micrometers, at least 40 micrometers, at least 50 micrometers, at least 75 micrometers, at least 100 micrometers, at least 200 micrometers, at least 300 micrometers, at least 500 micrometers, at least 1 mm, at least 2 mm, at least 3 mm, at least 5 mm, at least 10 cm, etc. Combinations of any of the above are also possible. For example, the droplet may have an average or characteristic diameter of between 40 micrometers and 100 micrometers, between 50 micrometers and 1 mm, between 1 mm and 1 cm, etc. As mentioned, if a plurality of droplets are present (for example, a target droplet and one or more other droplets that are in contact or in ionic communication with the target droplet), then the droplets may independently be the same or different sizes, diameters, or volumes, etc., including any of those discussed herein. For example, the target droplet may have substantially the same volume (e.g., within 20%, within 10%, or within 5% by volume), or different volumes than other droplets. For instance, the target droplet may be bigger or smaller than other droplets.

It should be understood that within the droplets, the charge carriers are predominately ions, rather than electrons. Thus, the droplets can be said to be in ionic communication with each other, e.g., such that ions can flow through or between droplets, thereby creating an electrical pathway between electrodes. In particular, without wishing to be bound by any theory, it is believed that when a voltage or current is applied, an electrochemical reaction occurs in which electrons are converted into ions at one electrode while ions are converted to electrons at the other electrode, thereby allowing the flow of charge carriers (i.e., current) and completing the electrical circuit.

However, the process of converting electron flow to ion flow (or vice versa) typically involves an electrochemical reaction, which can also result in chemical by-products near the electrodes. As a non-limiting example, water may react at the electrodes as follows:

2H₂O+2e⁻→H_{2(g)}+2OH⁻_{(aq)}

2OH⁻_{(aq)}→½O_{2(g)}+H₂O₍ₗ₎+2e⁻.

In this example, electrons are effectively converted into ions (OH) at the first electrode, while ions are converted into electrons at the second electrode. However, such chemical reactions also may cause the formation of by-products, pH changes, or the like near each electrode. For instance, in this example, by-products such as H₂ or O₂ may be created. However, as discussed herein, such chemical by-products that are produced at the electrodes may constrained using droplets surrounding the electrodes, thereby preventing or reducing the ability of such chemical reactions from affecting cells or other entities contained within a target droplet. For instance, one or both of the electrodes may be in contact with a respective droplet, which can be used to contain chemical by-products produced by the electrodes, thereby preventing them from reaching a target droplet.

As mentioned, current within droplets is typically carried by ions within the droplet, e.g., OH within the example above. Other ions (e.g., from ionic salts, such as Na⁺, K⁺, Cl⁻) may also carry charge as well within the droplets. Accordingly, a target droplet may be in ionic communication with electrodes via one or more other droplets creating a pathway through which current can flow (with ions acting as charge carriers within such droplets). As an illustrative non-limiting example, in FIG. 1, target droplet 15 is in ionic communication with electrodes 21 and 22 via droplets 11 and 12, with ions acting as charge carriers within droplets 11 and 12.

It should be understood that in some embodiments, the ions acting as charge carriers are not precisely known, e.g., since more than one ion may be present, although such ionic communication may nonetheless be determined, for example, as a current passing through the droplets or other portions of an electrical circuit. In addition, in certain embodiments, the droplets may be positioned such that there is a relatively even interface between the droplets, which may allow the voltage or the current to be essentially evenly applied along the interface.

Typically, a fluid containing ions and acting as a current pathway will have some degree of electrical resistance, and based on Ohm's law (V=IR), the voltage drop across such a fluid will be a function of the current and the resistance of the fluid. The resistance of the fluid may be controlled by factors such as the electrical resistivity of the fluid (e.g., based on the composition of the fluid), the length or distance that ions need to flow through the fluid, the cross-sectional area of the fluid pathway, etc. As discussed herein, these may be controlled in various embodiments to control the voltage experienced by the target droplet.

For example, in one set of embodiments, the target droplet may contain a fluid having a relatively high-resistivity media (or equivalently, a relatively low-conductivity media) while other droplets may contain fluids having a relatively low-resistivity media (or equivalently, a relatively high-conductivity media). (However, it should be understood that in other embodiments, the resistivity of the target droplet may be substantially equal to or even less than the resistivity of other droplets.) The voltage drops within various droplets, including the target droplet may be controlled by controlling, for example, the compositions of the fluids, the number of droplets, the length of the ionic communication pathway, etc. If more than one droplet is present, the sizes, compositions, etc. of the droplets may independently be the same or different. For example, by using droplets with fluids having different resistivities or conductivities, smaller or larger voltages may be created within the droplets. This may be useful, for example, to cause a larger voltage drop to be created in a target droplet (e.g., to cause electroporation in cells within the droplet) while smaller voltage drops may be created in the other (non-target) droplets. Thus, as a non-limiting example, referring again to FIG. 1, target droplet 15 may contain a relatively low-conductivity media while droplets 11 and 12 may contain relatively high-conductivity media (which may be the same or different).

In certain embodiments, if a plurality of droplets are present, e.g., a target droplet and one or more other droplets, the droplets may each independently have the same or different compositions. This may be useful, for example, to create different voltage drops in different droplets as mentioned (e.g., due to the droplets having different conductivities or resistivities created by the different compositions), or for other applications, including any of those described herein. In one set of embodiments, target droplets or other droplets having certain compositions, e.g., having relatively high conductivities or relatively low conductivities, may be added to a device, or created on a device, e.g., by mixing of other stock fluids to create a fluid with a desired conductivity. For example, as discussed in more detail herein, the device may be a digital microfluidic device, and various droplets may be created directly from source fluids, and/or may be created by combining other droplets together to produce droplets of a desired composition or of a desired conductivity, etc. In some cases, for instance, a target droplet may have a resistivity that is greater than the resistivity of other droplets, or a conductivity that is lower than the conductivity of other droplets.

In some embodiments, a droplet may contain a medium suitable for the cells, e.g., the droplet may contain cell culture media, serum, or other species suitable for maintaining cells. Non-limiting examples of cell culture media include DMEM, MEM, RPMI, PBS, or the like. In one example, the medium may contain an aqueous fluid, e.g., containing one or more salts. As another non-limiting example, a droplet may contain a medium suitable for electroporation, e.g., electroporation buffer. Electroporation buffers may mimic the composition of the cytoplasm composition, and a variety of such electroporation buffers can be obtained commercially. In addition, other examples of fluids that may be contained within droplets include, but are not limited to, water, saline, ethanol, or the like. In some embodiments, one or more salts may be dissolved in water to form an aqueous solution that may be contained within a droplet.

Other droplets within the device may have the same or different compositions than the target droplet. In some cases, for example, the other droplets may contain fluids having a lower resistivity or a higher conductivity than the fluid in the target droplet, or vice versa. Thus, as a non-limiting example, the target droplet may contain an electroporation buffer, while the other droplets may contain a fluid having higher conductivity than the electroporation buffer, for example, cell culture media, high-salt saline, or the like. In addition, according to certain embodiments, the conductivity ratio of the other droplets to the target droplet may be at least 1.5:1, at least 2:1, at least 3:1, at least 4:1, at least 5:1, at least 6:1, at least 8:1, at least 10:1, etc.

Non-limiting examples of fluids with relatively high conductivities or relatively low resistivities include, but are not limited to, saline or certain types of cell culture media such as DMEM, MEM, RPMI, etc. In some cases, the conductivity of a fluid having a relatively high conductivity may be at least 1 S/m, at least 1.5 S/m, at least 2 S/m, at least 2.5 S/m, at least 3 S/m, at least 3.5 S/m, at least 4 S/m, at least 4.5 S/m, at least 5 S/m, at least 10 S/m, etc. Non-limiting examples of fluids with relatively low conductivities or relatively high resistivities include, but are not limited to, electroporation buffer. In some cases, the conductivity of a fluid having a relatively low conductivity may be no more than 1 S/m, no more than 500 mS/m, no more than 300 mS/m, no more than 200 mS/m, no more than 100 mS/m, no more than 50 mS/m, no more than 30 mS/m, no more than 20 mS/m, no more than 10 mS/m, no more than 5 mS/m, no more than 3 mS/m, no more than 2 mS/m, no more than 1 mS/m, etc.

In one set of embodiments, a droplet may contain a medium having a relative permittivity or a dielectric constant of less than 60, less than 50, less than 40, or less than 30. In addition, in some embodiments, a medium in a non-target droplet may have a relative permittivity or a dielectric constant of at least 50, at least 60, at least 70, at least 80, or at least 90. The relative permittivity of the target droplet may be less than the relative permittivity of the non-target droplet. For example, the relative permittivity of the medium contained in a target droplet to the relative permittivity of the medium contained in a non-target droplet may be at least 1:1.5, at least 1:2, at least 1:2.5, or at least 1:3.

In addition, in one set of embodiments, a droplet, such as a target or other droplet, may independently contain oils or air, e.g., within the droplet, or forming a core-shell structure around the droplet. Non-limiting examples of oils that may be used include, but are not limited to, hydrocarbons, silicone oils, mineral oils, fluorocarbon oils, organic solvents, etc.

As mentioned, current may be applied to a plurality of droplets using one or more electrodes, which may be formed from suitable conductors. The electrodes may be in physical contact or be in ionic communication with a droplet, which may be in physical contact or ionic communication with other droplets (e.g., a target droplet).

In some embodiments, separating the electrodes from the target droplet, e.g., using fluids such as those contained within other droplets, may be useful for containing ions created at the electrodes, e.g., when the electrodes are used to create current or voltage. Thus, for example, a droplet surrounding an electrode may act as an ion containment system, generally preventing ions or other chemical by-products created at the electrodes from reaching a target droplet (or other droplets not in contact with the electrode), or from reaching cells or other entities contained within a target droplet. In some cases, such droplets may be present at some or all of the electrodes within the device that are used to produce a current or voltage, e.g., for electroporation or other purposes such as those described herein. The droplets used to contain ions or other chemical by-products from the electrodes may be in direct contact with the target droplet, or in some embodiments, there may be one or more intervening droplets or other fluids or materials.

The electrodes within the device may be made out of the same or different conductive materials, and may have the same or different shapes. Non-limiting examples of electrode materials include metals such as gold, silver, copper, platinum, steel, titanium, brass, palladium, or the like. Other materials may also be used in electrodes, for example, oxides (for example, titanium oxide, zirconium oxide, niobium oxide, tantalum oxide, ruthenium oxide, iridium oxide, platinum oxide, etc.), graphite, carbon, conductive polymers, or the like. It should be understood that since ion containment can be used in accordance with some embodiments to reduce or prevent contamination of a target droplet with ions or other chemical by-products created at the electrodes, a wide variety of materials can be used, including those that are known to create such by-products.

The electrode may have an interface that is of any suitable shape. Non-limiting examples of shapes include circular, rectangular, square, triangular, polygonal, irregular, or the like. In some cases, one or more electrodes may have an interface with a non-circular geometry. If more than one electrode is present, the electrode interfaces may have the same or different shapes. In some embodiments, the electrodes may have an interfacial shape that alters the geometry of a droplet, such as a target droplet. For instance, the electrode interface may have a shape that causes a droplet to become elongated, e.g., the interface may be an ellipse or an oval.

The electrodes may be positioned at any suitable location within the device. For example, the electrodes may be positioned in a substrate, for example, in a planar substrate, such as in a digital microfluidic device. The electrodes may independently be located in the same or different substrates, e.g., if more than one substrate is present. For example, in a digital microfluidic device, the electrodes may be integrated into a single substrate (e.g., a top substrate, a bottom substrate, etc.), or in more than one substrate (e.g., one in a first substrate and one in a second substrate). Other configurations are also possible in other embodiments. In addition, in some embodiments, one or more of the electrodes may be separate from the substrates. For example, the electrode may be present as a wire or other electrical connector that is separate or electrically isolated from a substrate. The electrodes may be positioned with any suitable spacing within the device. For example, the electrodes may be separated by at least 10 micrometers, at least 20 micrometers, at least 30 micrometers, at least 40 micrometers, at least 50 micrometers, at least 60 micrometers, at least 70 micrometers, at least 80 micrometers, at least 90 micrometers, at least 100 micrometers, at least 200 micrometers, at least 300 micrometers, at least 500 micrometers, at least 1 mm, at least 2 mm, at least 3 mm, at least 5 mm, at least 1 cm, at least 2 cm, at least 3 cm, at least 5 cm, etc. In some cases, the electrodes may be separated by no more than 5 cm, no more than 3 cm, no more than 2 cm, no more than 1 cm, no more than 5 mm, no more than 3 mm, no more than 2 mm, no more than 1 mm, no more than 500 micrometers, no more than 300 micrometers, no more than 200 micrometers, no more than 100 micrometers, no more than 50 micrometers, no more than 30 micrometers, no more than 20 micrometers, no more than 10 micrometers, etc. Combinations of any of these are also possible in some embodiments. For instance, the electrodes may be separated by a distance of between 20 micrometers and 30 micrometers, between 50 micrometers and 100 micrometers, between 1 mm and 1 cm, or the like. However, it should be understood that the physical distance between the electrodes need not necessarily define the length of the ionic communication pathway created between the electrodes when voltage is applied. For instance, a chain of droplets defining the ionic communication pathway between the electrodes may be non-linear, thereby defining a longer distance for current to flow.

In some cases, one or more of the electrodes may be coated, for example, with a hydrophilic coating. If more than one electrode is present, the electrodes may independently be coated or uncoated, and/or having the same or different coatings. Non-limiting examples of suitable coating materials, include, but are not limited to, silane coatings. In some embodiments, reagents or other materials may be attached to the coatings, e.g., chemically or physically. As a non-limiting example, reagents such as sgRNA or guide RNA may be attached to an electrode, e.g., for applications involving inserting biomolecules (such as CRISPR-associated ribonucleoproteins, genes such as gRNA or Cas9, etc.) into living cells via electroporation, for instance, for gene editing.

In some cases, the voltage applied to the electrodes may be at least 10 V, at least 20 V, at least 30 V, at least 50 V, at least 100 V, at least 200 V, at least 300 V, at least 500 V, at least 1 kV, etc. In some embodiments, the voltage applied to the electrodes may be no more than 1 kV, no more than 500 V, no more than 300 V, no more than 200 V, no more than 100 V, no more than 50 V, no more than 30 V, no more than 20 V, no more than 10 V, etc. Combinations of any of these are also possible in certain embodiments, e.g., the voltage that is applied may be between 50 V and 200 V, between 500 V and 1 kV, between 10 V and 20 V, etc.

In addition, in certain cases, the voltage applied to the electrodes may be used to produce a voltage gradient within a target droplet of at least 1 V/cm, at least 2 V/cm, at least 3 V/cm, at least 5 V/cm, at least 10 V/cm, at least 20 V/cm, at least 30 V/cm, at least 50 V/cm, at least 100 V/cm, at least 200 V/cm, at least 300 V/cm, at least 500 V/cm, at least 1 kV/cm, at least 2 kV/cm, at least 3 kV/cm, at least 5 kV/cm, etc. In some cases, the target droplet may experience a voltage gradient of no more than 5 kV/cm, no more than 3 kV/cm, no more than 2 kV/cm, no more than 1 kV/cm, no more than 500 V/cm, no more than 300 V/cm, no more than 200 V/cm, no more than 100 V/cm, no more than 50 V/cm, no more than 30 V/cm, no more than 20 V/cm, no more than 10 V/cm, no more than 5 V/cm, no more than 3 V/cm, no more than 2 V/cm, no more than 1 V/cm, etc. In addition, in some cases, combinations of any of these may be used, e.g., the voltage applied to the electrodes that in used to produce a voltage gradient within a target droplet may be between 10 V/cm and 50 V/cm, between 50 V/cm and 100 V/cm, between 500 V/cm and 2 kV/cm, etc. Additionally, in some embodiments, the voltage or voltage gradient crated in a target droplet may be relatively uniform or homogenous. For instance, by using a fluid with a relatively high conductivity, a relatively uniform or homogenous electric field may be created within the target droplet. As a non-limiting example, a figure showing the electric field distribution in a target droplet adjacent to two other droplets is shown in FIG. 9.

A variety of devices and techniques may be used to produce droplet configurations such as those described herein. For example, in some cases, the droplets may be manually positioned between electrodes. In addition, in certain embodiments, a digital microfluidic device may be used to position droplets to connect between electrodes, e.g., able to apply voltages or currents such as are described herein to a target droplet. In a digital microfluidic device, droplets may be present on a substrate, e.g., a planar substrate, and moved around the substrate using techniques such as electrowetting, dielectrophoresis, immiscible-fluid flow, or the like.

A variety of electrodes may be present to manipulate or facilitate the movement of such droplets on the substrate, often defining a plurality of “pixels” or locations on the substrate where a droplet may be present. Those of ordinary skill in the art will be familiar with a variety of digital microfluidic devices and techniques for manipulating droplets therein, including moving, sorting, merging, mixing, splitting, etc. such droplets. In addition, it should be understood that the “droplets” within a digital microfluidic device are not necessarily spherical or circular, but may adopt a variety of other forms and shapes, for example, as defined by the pixels within the device.

Accordingly, in some embodiments, for instance, droplets, including a target droplet, may be moved into position between electrodes in the digital microfluidic device, for example, creating an ionic communication pathway between the electrodes, then a voltage or current applied using the electrodes to the target droplet. The digital microfluidic device may have 2, 3, 4, or more such electrodes able to supply voltages or currents to the target droplet, e.g., to cause electroporation within cells within a target droplet, and such electrodes may be the same as those used to manipulate droplets on the substrate, or different.

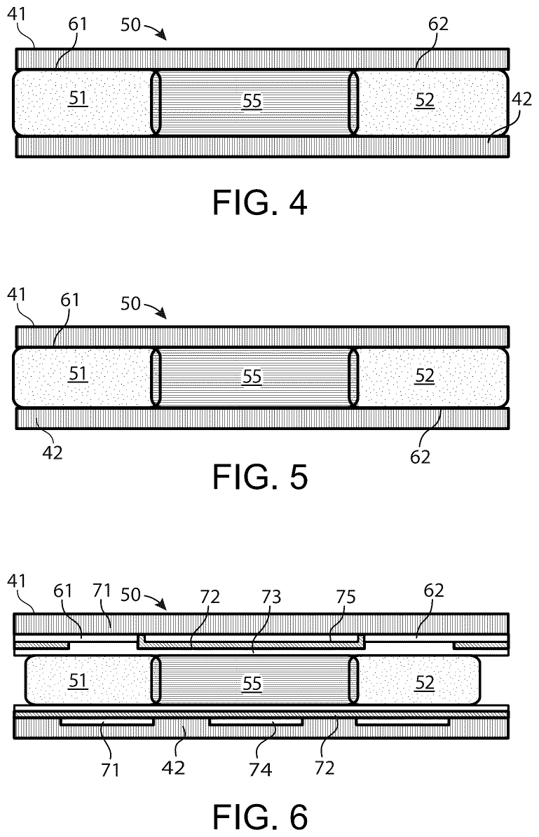

In addition, in some cases, a digital microfluidic device may include parallel plates or substrates in which the droplets of fluid are contained and manipulated between, and in some cases, the electrodes used for applying relatively high voltages (e.g., to cause electroporation, or other applications such as those described herein) may each be in the same plate or substrate, or in different plates or substrates. Non-limiting examples of such configurations can be seen in FIGS. 4-6.

In some cases, droplets within a digital microfluidic device may be controlled to control a target droplet. For instance, a target droplet, and/or other droplets may be controlled within the digital microfluidic device to cause a certain voltage to be applied to the target droplet. Parameters such as the number of droplets, the compositions of the droplets, the positions of the droplets, the sizes of the droplets, the volume of the droplets, the electrical resistances of the droplets, etc. may be readily controlled using techniques for manipulating droplets within a digital microfluidic device, such as those previously discussed.

For instance, in certain embodiments, a target droplet may be moved within the digital microfluidic device to a first location, while other droplets may be moved to other locations (e.g., to be disposed of as waste). As a non-limiting example, if the target droplet contains cells, after applying a voltage (e.g., to cause electroporation within the cells), the target droplet may be moved within the digital microfluidic device to a location where the cells are allowed to recover. In some cases, for example, the droplets containing the cells may be combined with droplets containing recovery buffer or cell media, etc.

It should be understood that in some embodiments, more than one target droplet may be manipulated, e.g., sequentially and/or simultaneously. For instance, a device, such as a digital microfluidic device may have a plurality of target droplets, e.g., to which a voltage is to be applied (e.g., to cause electroporation within cells which may be present in the target droplets). The droplets may be manipulated such that they are also exposed to the same electrodes, and/or two different electrodes. For example, a voltage may be applied to a first target droplet as discussed herein, then the first target droplet moved away from the electrodes and a second target droplet moved into that location. As another non-limiting example, a first voltage may be applied to a first target droplet using a first pair of electrodes and a second voltage may be applied to a second target droplet using a second pair of electrodes, e.g., simultaneously and/or sequentially with respect to the first target droplet.

In certain embodiments, after a voltage or current has been applied to a target droplet, e.g., as discussed herein, the target droplet may be moved away from the electrodes and/or from other droplets. This may be useful, for example, to prevent or reduce contamination of the target droplet by the other droplets and/or from the electrodes. In addition, as mentioned, a target droplet may be moved in certain embodiments to a location where cells that may be present within the target droplet are allowed to recover, e.g., after current and/or voltage has been applied, e.g., as in an electrical pulse used to cause electroporation within the cells.

U.S. Provisional Patent Application Ser. No. 63/197,202, filed Jun. 4, 2021, entitled “Systems and Methods for Applying Voltages within Droplet-Based Systems,” by Shih, et al., is incorporated herein by reference in its entirety.

The following examples are intended to illustrate certain embodiments of the present disclosure, but do not exemplify the full scope of the disclosure.

### Example 1

This example illustrates a device for inserting high value biomolecules (such as, for example, CRISPR associated ribonucleoproteins, nucleases capable of gene editing into mammalian cells, etc.) into living cells via electroporation with high viability and high efficiency, in accordance with certain embodiments. The device in this example is able to isolate cells from harmful by-products of electroporation while exposing the cells to a homogenous electric field. It can also facilitate placement of cells into recovery buffer. The device described in this example can also be modified to facilitate lower voltages or varying cell amounts. This device can handle extremely low quantities of cells (a functionality lacking in currently available devices). Examples of cells that this device could be used for include, but are not limited to, mammalian cells or microbial cells.

The device described in this example is an electroporation system where the anode and cathode are electrically connected by a chain of 3 or more droplets. The outer two droplets may comprise a high-conductivity media, and the middle droplet may comprise a low-conductivity electroporation buffer containing cells and biomolecules to be inserted into the cells. See, e.g., FIG. 1.

By placing a high conductivity droplet in contact with the anode and cathode, these droplets are essentially transformed into “liquid electrodes,” and the middle droplet acts as a “liquid cuvette.” Forming the electroporation apparatus out of liquid droplets provides certain advantages, such as the following.

The biological materials contained in the “liquid cuvette” (i.e., in the middle droplet) are isolated from the electrodes. This may protect sensitive materials within the middle droplet from the effects of electrolysis at the boundary between the electrodes and the “liquid electrodes” (i.e., the outer droplets).

The “cuvette” geometry, or the dimensions of the liquid containing the cells, may be defined by the droplets in the droplet chain. Thus, in certain embodiments, the voltage required to get a certain electric field can be reduced or increased as desired, for example, by controlling the droplets within the droplet chain, e.g., by controlling their number, size, composition, electrical resistance, or the like. For instance, in one set of embodiments, the distance between the anode and the cathode through a pathway of one or more droplets may be controlled, e.g., to “narrow” or “widen” the “liquid cuvette.”

The electrical resistance of the droplet chain may depend on factors such as the distance between the anode and the cathode, or the cross-sectional area of the droplet chain. This may affect the voltage that is applied. The cross-sectional area may vary based on factors such as the geometry of the droplet chain, e.g., its width, height, and/or length.

The high conductivity of the droplets (i.e., the “liquid electrodes”) may allow for the voltage to be essentially evenly applied along the interface between those droplets and the droplets containing cells (i.e., the “liquid cuvette”). In some cases, the resulting electric field may be homogenous applied across the droplet containing cells. This can stand in contrast to other electrode systems, where the electric field is highly localized to the plane on which the electrodes are located, resulting in poor electric field distribution, e.g., applied to the cells.

In addition, in certain embodiments, droplets containing cells can be mixed with a recovery buffer, e.g., immediately after electroporation.

The above system can be integrated in some embodiments into a digital microfluidic (DMF) device. For example, the anode and the cathode can be integrated into either a top plate, a bottom plate, or one on the top plate and one on the bottom plate. Other configurations, including those described herein, are also possible. This may allow for droplets to be placed and manipulated, for example, via electrowetting actuation, for droplets to be introduced to an electroporation site via techniques such as electrowetting-on-dielectric (EWOD), for droplets to be added or removed as needed, or the like.

FIG. 1 illustrates three droplets in a row in device 10, making a continuous electrically connected chain. The outer two droplets 11, 12 may contain media with a relatively high electrical conductivity, while middle droplet 15 may contain a media having a relatively low electrical conductivity. The outer two droplets are in contact with conductors 21, 22 controlled by electrical pulse generator 30. FIG. 2 is similar to FIG. 1, except that the droplets are present on substrate 40. FIG. 3 is also similar, except that in this figure, conductors 21 and 22 are contained within substrate 40.

In FIGS. 4 and 5, three droplets are illustrated in a row within device 50, making a continuous electrically connected chain. The outer two droplets 51, 52 may contain a media with a relatively high electrical conductivity, and middle droplet 55 may contain a media with a relatively low electrical conductivity. The droplets in this figure are sandwiched between two substrates 41, 42. Conductors may be integrated into either substrate or inserted into the droplets directly, etc. In FIG. 4, conductors 61 and 62 are each integrated within substrate 41, while in FIG. 5, conductor 61 is contained within substrate 41 while conductor 62 is contained within substrate 42.

FIG. 6 illustrates three droplets sandwiched between two substrates 41, 42. These substrates can be used, for example, for EWOD based actuation. The outer two droplets 51, 52 may contain a relatively high conductivity media, while middle droplet 55 may contain a relatively low conductivity media. The outer two droplets are in contact with electrodes 61, 62, which may be contained within substrate 41. In this example, substrate 41 may contain a glass layer 71, a dielectric layer 72, a hydrophobic layer 73, electrodes 74 (e.g., for performing DMF), and electrodes 75 (e.g., for applying a voltage to cause electroporation). As a non-limiting example, electrode 74 may comprise chromium and/or electrode 75 may comprise gold.

In some cases, the target droplet may include a high resistance fluid, such as a 1 M electroporation buffer. One non-limiting example of an electroporation buffer is an aqueous solution of the following: 5 mM KCl, 15 mM MgCl₂, 120 mM Na₂HPO₄/NaH₂PO₄, and 50 mM mannitol at a pH of 7.2.

### Example 2

This non-limiting example presents various experiments illustrating electroporation in droplets, in accordance with certain embodiments of the invention.

Experimental overview: in these experiments, droplet operation was composed of four key steps: (1) reservoir filling. (2) tri-droplet dispensing, (3) tri-droplet merging, and (4) tri-droplet electroporation. The device had three reservoirs: two outer reservoirs were filled with PBS (or high conductivity buffer, σ (sigma)˜16 mS/cm) and the middle reservoir was filled with cells and the desired payload suspended in low conductivity electroporation buffer (σ (sigma)˜8.4 mS/cm). Reservoirs were filled by pipetting 6 microliters each onto the bottom plate at the edge of the top plate and actuating 3 reservoir electrodes with a Digital Microfluidic (DMF) driving potential (300 Vᵣₘₛ and 15 kHz) to draw the fluids into the reservoir. Next, a 1 microliter single droplet was dispensed from each reservoir using DMF actuation to pull the liquid out of the reservoir and implementing an on-chip droplet dispensing technique. The cell containing droplet was actuated to the center of the electroporation site and the two PBS dispensed droplets were actuated to the outer edges of the electroporation site. The three droplets were merged by actuating the PBS droplets inwards, towards the cell containing droplet creating a continuous tri-droplet structure. Immediately upon merging, the electroporation circuit was triggered to deliver a sequence of three high voltage DC (0-250 V) square-wave pulses (10 ms) to the exposed Au-electrodes that were in direct contact with the PBS droplets. After electroporation, the top plate was removed, and the electroporated cells were immediately placed in a 96 well plate that was pre-loaded with 150 microliters of warmed complete culture media with no antibiotics.

For all experiments described herein the cell line was HEK293 and the delivered payload was a dextran molecule of various sizes (70 kDa, 25 0 kDa, 2000 kDa) conjugated with a FITC molecule for fluorescent detection. 300 micrograms/ml of dextran was added regardless of size.

Transfection efficiency and viability was measured using a BD FACS Melody (BD Bioscience, Canada). Prior to FACS, cells were resuspended in culture media, and then washed by centrifuging (300 g, 3 minutes) and resuspending in 1 ml of PBS, then centrifuged again (300 g, 3 minutes) and resuspended in 600 microliters of FACS buffer. Viability was assessed using DAPI added immediately prior to FACS.

Neon Type T EP buffer was used as the low conductive buffer in all experiments. 0.05% F68 surfactant was added at 8.4 mS/cm. PBS was used as the high conductive buffer in all experiments. 0.05% F68 surfactant was added at 16.0 mS/cm. The payload was FITC-tagged dextran molecules of various sizes. Live/Dead staining was performed using DAPI.

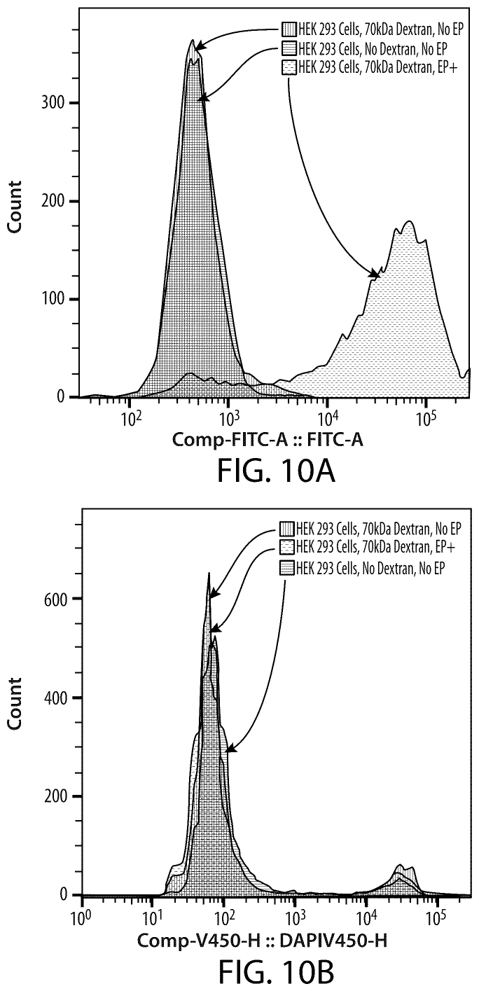

FIGS. 10A-10B show a comparison of “Tridrop” (three droplet) electroporation and controls. FIG. 10A shows the FITC expression profiles of cells exposed to three conditions. The first condition (No Dextran, No EP) shows the FITC expression profile of cells, untreated with dextran, that remained in culture media for the duration of the experiment. The second condition (70 kDa Dextran, No EP) shows the expression profile of cells with 70 kDa dextran that were in electroporation buffer for ˜10 minutes but were not exposed to on-chip electroporation. The final condition (70 kDa Dextran, EP+) shows the expression profile of cells with 70 kDa dextran that were exposed to Tridrop electroporation (3, 200 V, 10 ms pulses). FIG. 10B shows the DAPI expression profile of all three conditions, which suggest that using Tridrop electroporation can significantly increase FITC uptake while having a minimal effect on cell viability.

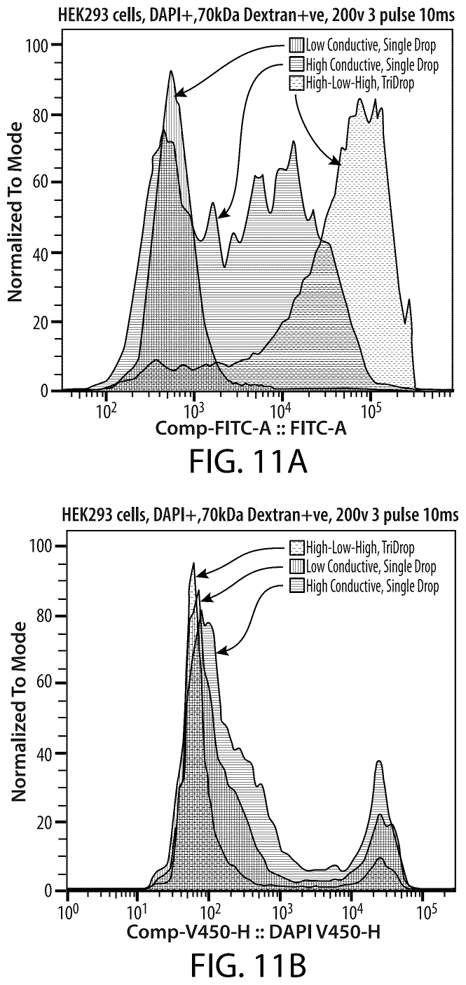

FIGS. 11A-11B show the importance of high-low-high drop configuration. FIG. 11A shows the FITC expression profile. FIG. 11B shows DAPI expression profile for three conditions containing 70 kDa dextran all electroporated with 3, 200 V, 10 ms pulses. The first condition (Low Conductive, Single Drop) shows the results of electroporation when all three droplets in the tridrop structure are comprised of low conductivity media. The second condition (High Conductive, Single Drop) shows the results of electroporation when all three droplets in the tridrop structure are comprised of high conductivity media. The final condition (High-Low-High, TriDrop) shows the results of electroporation when all the outer droplets in the tridrop structure are comprised of high conductivity media and the inner droplet is comprised of low conductivity media. These results show that the high-low-high droplet structure allows the electric field to be focused into the central droplet providing a significant electroporating force while preserving cell health.

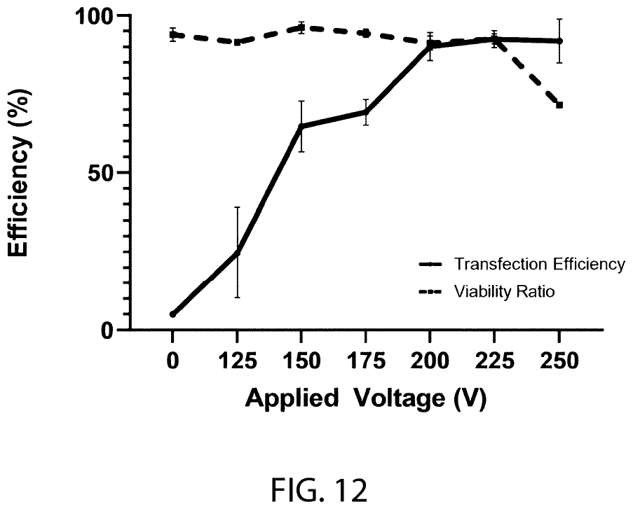

FIG. 12 shows voltage optimization. The transfection efficiency (solid line) and viability ratio (dashed line) are shown with increasing voltage. 70 kDa dextran was used. Viability ratio is defined as the viability of a transfected population divided by the viability of a healthy untreated population. These results showed the optimal voltage range for performing tridrop electroporation. All samples treated with 3 pulses, 10 ms. Payload: FITC tagged 70 kDa Dextran, DAPI+.

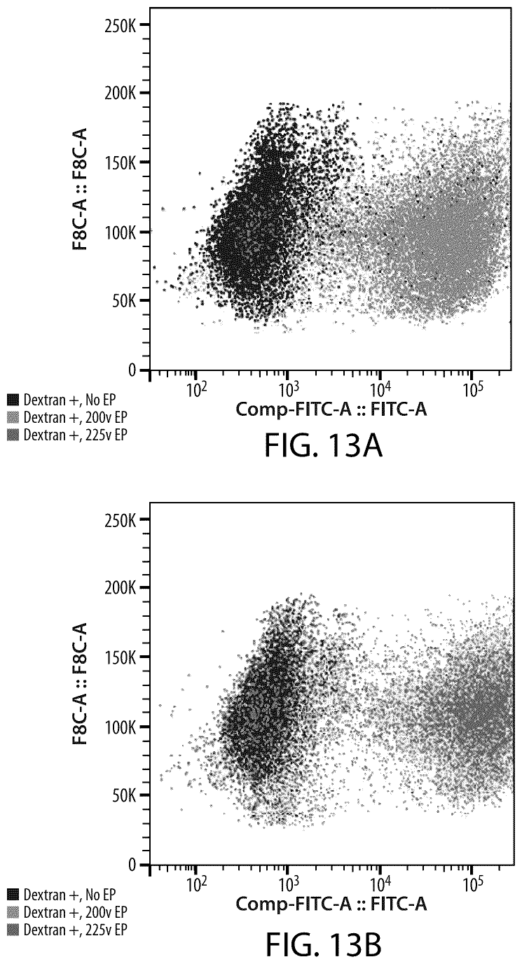

FIGS. 13A-13B show voltage optimization example data. Dot plots (Forward Scatter vs. FITC expression) are shown for 3 conditions, all containing 70 kDa dextran. The first condition shows un-electroporated cells (generally to the left in FIGS. 13A and 13B). The second condition shows cells electroporated with 3, 200 V, 10 ms pulses (generally to the right in FIG. 13A). The final condition shows cells electroporate with 3, 225 V, 10 ms pulses (generally to the right in FIG. 13B). Data shown treated with 3 pulses. 10 ms. Payload: FITC tagged 70 k Da Dextran, DAPI+.

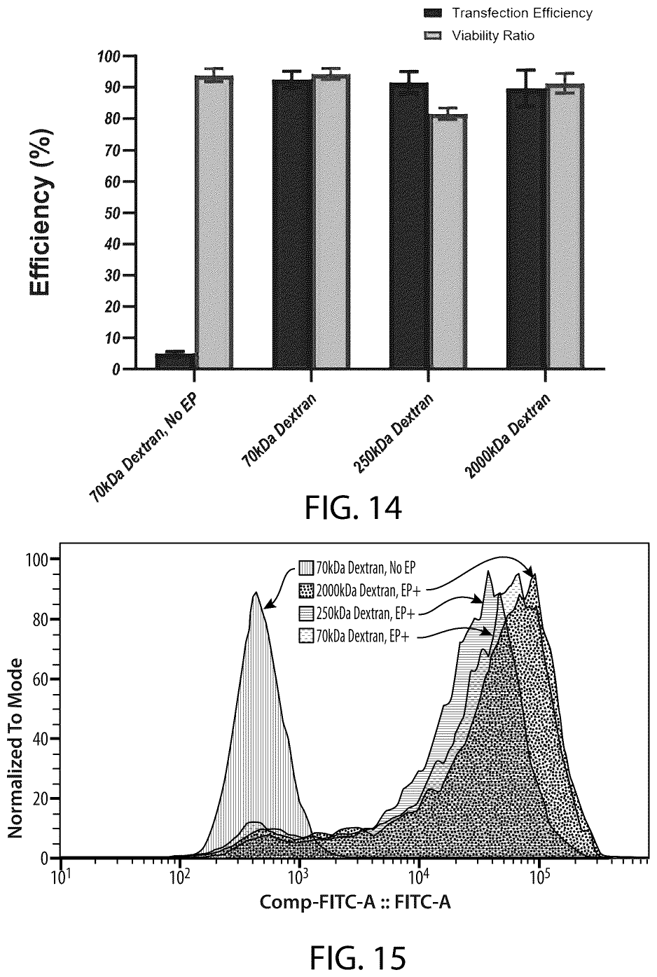

FIG. 14 shows tridrop payload testing. Transfection efficiency and viability ratio are shown when inserting dextran molecules of three different sizes (70 k Da, 250 kDa, and 2000 kDa) as well as an un-electroporated control. All samples were electroporated with 3, 200 V, 10 ms pulses. Dextran was added to a concentration of 300 micrograms/ml. These results suggest that tridrop electroporation can be used to insert large molecules into mammalian cells with minimal impact on cell health.

FIG. 15 shows FITC expression in tridrop payload testing. FITC expression of three different payloads inserted using Tridrop electroporation is shown. The first profile (70 kDa Dextran, no EP) shows un-electroporated cells with 70 kDa Dextran. The next three profiles show cells electroporated with 3 200 V, 10 ms pulses containing 70 kDa dextran (70 kDa Dextrna, EP+), 250 kDa dextran (250 kDa Dextran, EP+), and 2000 kDa dextran (2000 kDa Dextran, EP+). All samples treated with 200 V, 3 pulses, 10 ms. Payload: FITC tagged 70 kDa Dextran, DAPI+. These results suggest that not only are large dextran molecules being inserted efficiently and with minimal viability consequences, but that the cells are fluorescing with a similar intensity to that of cells that had a smaller molecule inserted into it.

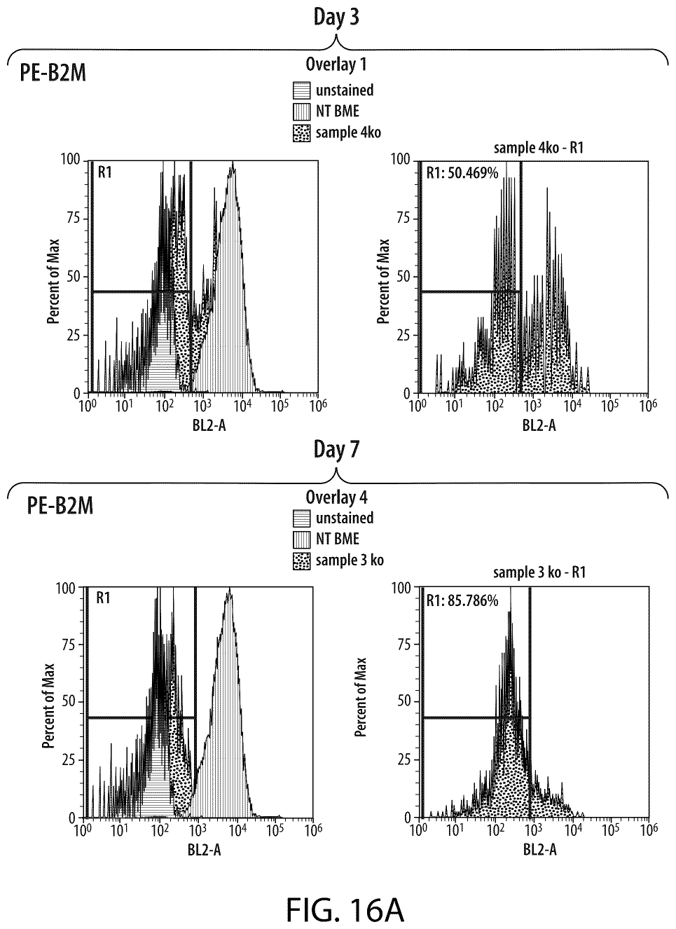

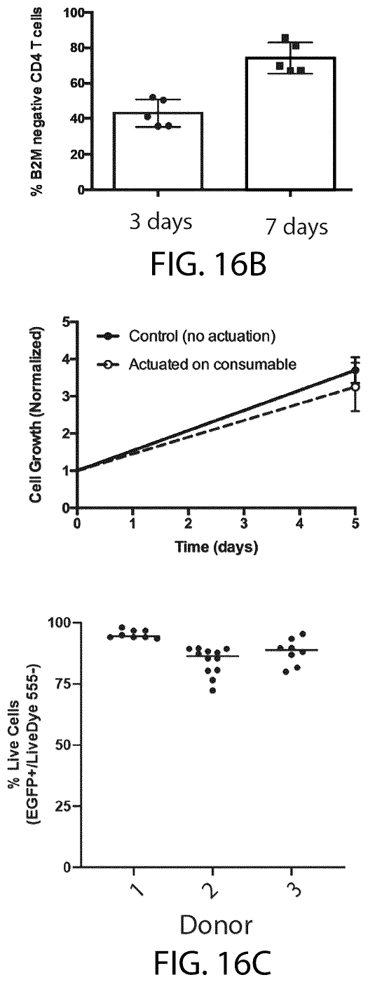

FIGS. 16A-16C show the editing efficiency and cell viability in primary human T cell populations using the tridrop method. FIG. 16A shows histograms illustrating the level of B2M at 3 days and 5 days in primary CD4+ T cells in non-electroporated and unstained (Unstained control), electroporated with sgRNA targeting B2M (B2M sgRNA (knock out)), or electroporated with sgRNA targeting scrambled sequences (scrambled sgRNA (control)). CD4+ T cells were isolated from the peripheral blood of human donors via positive selection. All data was captured using a flow cytometer (Attune, Thermo Fischer). Cas9 nuclease (Aldevron) was combined with synthetic guide (sg) RNA (Synthego) to form a ribonucleic protein (RNP) prior to delivery into cells. FIG. 16B shows the quantification of B2M levels in CD4+ human T cells at 3 days or 5 days post edit using the tridrop system. Percent negative B2M cells were quantified using flow cytometry (Attune, Thermo Fischer). Experiments were performed in technical duplicate or triplicate, across two human donors. FIG. 16C shows the viability of human T cells post electroporation with GFP mRNA (Tri Link). Cells were enumerated via flow cytometry at day 1 and day 5 post electroporation using Live/Dye 555 stain (Thermo Fischer). Data is representative of technical quadruplicate (upper panel). Cell viability was assessed after 5 days post electroporation. N=7, from 3 different human donors.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
contacting a target fluidic droplet with a first fluidic droplet in ionic communication with a first electrode and a second fluidic droplet in ionic communication with a second electrode; and
applying a voltage between the first electrode and the second electrode.

2. The method of claim 1, wherein the first fluidic droplet is in physical contact with the first electrode.

3. The method of any one of claim 1 or 2, wherein the second fluidic droplet is in physical contact with the second electrode.

4. The method of any one of claims 1-3, wherein the first fluidic droplet is in ionic communication with the first electrode via an ionic communication pathway extending through at least one intervening fluidic droplet between the first fluidic droplet and the first electrode.

5. The method of any one of claims 1-4, wherein the second fluidic droplet is in ionic communication with the second electrode via an ionic communication pathway extending through at least one intervening fluidic droplet between the second fluidic droplet and the second electrode.

6. The method of any one of claims 1-5, wherein the target fluidic droplet, the first fluidic droplet, and the second fluidic droplet are each present on a digital microfluidic device.

7. The method of claim 6, wherein the target fluidic droplet, the first fluidic droplet, and the second fluidic droplet are each defined by pixels on the digital microfluidic device.

8. The method of any one of claims 1-7, wherein the target fluidic droplet contains one or more cells.

9. The method of claim 8, wherein the applied voltage is at least sufficient to fuse the target fluidic droplet with the first fluidic droplet and/or the second fluidic droplet.

10. The method of any one of claim 8 or 9, wherein the applied voltage is at least sufficient to electroporate the one or more cells within the target fluidic droplet.

11. The method of claim 10, further comprising inserting molecules into the one or more cells after electroporating the one or more cells.

12. The method of claim 11, wherein the inserted molecules comprise nucleic acids.

13. The method of any one of claim 11 or 12, wherein the nucleic acids comprise ribonucleic acids.

14. The method of claim 13, wherein the ribonucleic acids comprise sgRNA.

15. The method of any one of claim 13 or 14, wherein the ribonucleic acids comprise guide RNA.

16. The method of any one of claims 1-15, comprising applying a voltage of at least 10 V between the first electrode and the second electrode.

17. The method of any one of claims 1-16, comprising applying a voltage of at least 50 V between the first electrode and the second electrode.

18. The method of any one of claims 1-17, comprising applying a voltage of at least 100 V between the first electrode and the second electrode.

19. The method of any one of claims 1-18, comprising applying a voltage of no more than 1 kV between the first electrode and the second electrode.

20. The method of any one of claims 1-19, comprising applying a voltage of no more than 500 V between the first electrode and the second electrode.

21. The method of any one of claims 1-20, wherein the target fluidic droplet experiences a voltage of at least 50 V when the voltage is applied between the first electrode and the second electrode.

22. The method of any one of claims 1-21, wherein the target fluidic droplet experiences a voltage of at least 100 V when the voltage is applied between the first electrode and the second electrode.

23. The method of any one of claims 1-22, wherein the target fluidic droplet experiences a voltage of at least 500 V when the voltage is applied between the first electrode and the second electrode.

24. The method of any one of claims 1-23, wherein the target fluidic droplet experiences a voltage gradient of at least 10 kV/cm when the voltage is applied between the first electrode and the second electrode.

25. The method of any one of claims 1-24, wherein the first fluidic droplet has an ionic conductivity of at least 1 S/m.

26. The method of any one of claims 1-25, wherein the second fluidic droplet has an ionic conductivity of at least 1 S/m.

27. The method of any one of claims 1-26, wherein the target fluidic droplet has an ionic conductivity of no more than 500 mS/m.

28. The method of any one of claims 1-27, wherein the conductivity of the first fluidic droplet is greater than the conductivity of the target fluidic droplet.

29. The method of any one of claims 1-28, wherein the conductivity of the first fluidic droplet is substantially equal to the conductivity of the second fluidic droplet.

30. The method of any one of claims 1-29, wherein the target fluidic droplet has substantially the same volume as the first fluidic droplet or the second fluidic droplet.

31. The method of any one of claims 1-29, wherein the target fluidic droplet has a bigger volume than the first fluidic droplet or the second fluidic droplet.

32. The method of any one of claims 1-31, wherein the first electrode and the second electrode are in a common substrate.

33. The method of any one of claims 1-31, wherein the first electrode is in a first substrate and the second electrode is in a second substrate.

34. The method of any one of claims 1-33, wherein the first electrode has a first interface and the second electrode has a second interface.

35. The method of claim 34, wherein the first interface is circular.

36. The method of claim 34, wherein the first interface is non-circular.

37. The method of any one of claims 34-36, wherein the first interface and the second interface have substantially the same shape.

38. The method of any one of claims 34-36, wherein the first interface and the second interface have substantially different shapes.

39. The method of any one of claims 1-38, further comprising moving the target fluidic droplet away from the first fluid droplet and the second fluidic droplet after applying the voltage.

40. The method of claim 39, further comprising moving a second target fluidic droplet to contact the first fluidic droplet in ionic communication with the first electrode and the second fluidic droplet in ionic communication with the second electrode, and thereafter applying a voltage between the first electrode and the second electrode.

41. The method of any one of claims 1-40, comprising applying the voltage between the first electrode and the second electrode as a first voltage pulse, the method further comprising applying a second voltage pulse between the first electrode and the second electrode after applying the first voltage pulse.

42. A method, comprising:
applying a voltage of at least 10 V to a target fluidic droplet using a first fluidic droplet and a second fluidic droplet, each in contact with the fluidic droplet.

43. The method of claim 42, wherein the target fluidic droplet, the first fluidic droplet, and the second fluidic droplet are each present on a digital microfluidic device.

44. The method of claim 43, wherein the target fluidic droplet, the first fluidic droplet, and the second fluidic droplet are each defined by pixels on the digital microfluidic device.

45. The method of any one of claims 42-44, wherein the applied voltage is at least sufficient to fuse the target fluidic droplet with the first fluidic droplet and/or the second fluidic droplet.

46. The method of any one of claims 42-45, wherein the target fluidic droplet contains one or more cells.

47. The method of claim 25, wherein the applied voltage is at least sufficient to electroporate the one or more cells within the target fluidic droplet.

48. The method of claim 47, further comprising inserting molecules into the one or more cells after electroporating the one or more cells.

49. The method of claim 48, wherein the inserted molecules comprise nucleic acids.

50. The method of claim 49, wherein the nucleic acids comprise ribonucleic acids.

51. The method of claim 50, wherein the ribonucleic acids comprise sgRNA.

52. The method of any one of claim 50 or 51, wherein the ribonucleic acids comprise guide RNA.

53. The method of any one of claims 42-52, comprising applying a voltage of at least 100 V to the target fluidic droplet.

54. The method of any one of claims 42-53, comprising applying a voltage of no more than 1 kV to the target fluidic droplet.

55. The method of any one of claims 42-54, wherein the target fluidic droplet has substantially the same volume as the first fluidic droplet or the second fluidic droplet.

56. The method of any one of claims 42-55, wherein the target fluidic droplet has a bigger volume than the first fluidic droplet or the second fluidic droplet.

57. The method of any one of claims 42-56, further comprising moving the target fluidic droplet away from the first fluid droplet and the second fluidic droplet after applying the voltage.

58. A digital microfluidic device, comprising:
a plurality of pixels, including a first pixel, a second pixel, and at least one pixel between the first pixel and the second pixel;
a first electrode in contact with the first pixel;
a second electrode in contact with the second pixel; and
a voltage generator able to produce a voltage of at least 10 V between the first electrode and the second electrode.

59. The digital microfluidic device of claim 58, wherein the first pixel, the second pixel, and the at least one pixel are colinear.

60. The digital microfluidic device of claim 58, wherein the first pixel, the second pixel, and the at least one pixel are not colinear.

61. The digital microfluidic device of any one of claims 58-60, wherein the second electrode is separated from the first electrode by at least 40 micrometers.

62. The digital microfluidic device of any one of claims 58-61, wherein the second electrode is separated from the first electrode by at least 100 micrometers.

63. The digital microfluidic device of any one of claims 58-62, wherein the second electrode is separated from the first electrode by at least 1 mm.

64. The digital microfluidic device of any one of claims 58-63, wherein the second electrode is separated from the first electrode by at least 1 cm.

65. The digital microfluidic device of any one of claims 58-64, wherein the first electrode and the second electrode are in a common substrate.

66. The digital microfluidic device of any one of claims 58-64, wherein the first electrode is in a first substrate and the second electrode is in a second substrate.

67. The digital microfluidic device of any one of claims 58-66, wherein the first electrode has a first interface and the second electrode has a second interface.

68. The digital microfluidic device of claim 67, wherein the first interface is circular.

69. The digital microfluidic device of claim 67, wherein the first interface is non-circular.

70. The digital microfluidic device of any one of claims 67-69, wherein the first interface and the second interface have substantially the same shape.

71. The digital microfluidic device of any one of claims 67-69, wherein the first interface and the second interface have substantially different shapes.

72. The digital microfluidic device of any one of claims 58-71, wherein the voltage generator is able to produce a voltage of at least 50 V.

73. A digital microfluidic device, comprising:
a first electrode;
a second electrode separated from the first electrode by at least 10 micrometers; and
a voltage generator able to produce a voltage of at least 10 V between the first electrode and the second electrode.

74. The digital microfluidic device of claim 73, wherein the second electrode is separated from the first electrode by at least 40 micrometers.

75. The digital microfluidic device of any one of claim 73 or 74, wherein the second electrode is separated from the first electrode by at least 100 micrometers.

76. The digital microfluidic device of any one of claims 73-75, wherein the second electrode is separated from the first electrode by at least 1 mm.

77. The digital microfluidic device of any one of claims 73-76, wherein the second electrode is separated from the first electrode by at least 1 cm.

78. The digital microfluidic device of any one of claims 73-77, wherein the first electrode and the second electrode are in a common substrate.

79. The digital microfluidic device of any one of claims 73-77, wherein the first electrode is in a first substrate and the second electrode is in a second substrate.

80. The digital microfluidic device of any one of claims 73-79, wherein the first electrode has a first interface and the second electrode has a second interface.

81. The digital microfluidic device of claim 80, wherein the first interface is circular.

82. The digital microfluidic device of claim 80, wherein the first interface is non-circular.

83. The digital microfluidic device of any one of claims 80-82, wherein the first interface and the second interface have substantially the same shape.

84. The digital microfluidic device of any one of claims 80-82, wherein the first interface and the second interface have substantially different shapes.

85. The digital microfluidic device of any one of claims 73-84, wherein the voltage generator is able to produce a voltage of at least 50 V.

86. An electroporation system, comprising:
a first ion containment system surrounding a first electrode;
a second ion containment system surround a second electrode; and
a target fluidic droplet in electrical communication with the first electrode and the second electrode,
wherein, when a voltage is applied between the first electrode and the second electrode, ions created at each of the first electrode and the second electrode are contained in the respective first and second ion containment systems.

87. The electroporation system of claim 86, wherein the electroporation system is a digital microfluidic device.

88. The electroporation system of any one of claim 86 or 87, wherein the first ion containment system comprises a first fluidic droplet surrounding the first electrode.

89. The electroporation system of any one of claims 86-88, wherein the second ion containment system comprises a second fluidic droplet surrounding the second electrode.

90. The electroporation system of any one of claims 86-89, wherein the target fluidic droplet contains one or more cells.

91. The electroporation system of any one of claims 86-90, wherein the first ion containment system has an ionic conductivity of at least 1 S/m.

92. The electroporation system of any one of claims 86-91, wherein the second ion containment system has an ionic conductivity of at least 1 S/m.

93. The electroporation system of any one of claims 86-92, wherein the target fluidic droplet has an ionic conductivity of no more than 500 mS/m.

94. The electroporation system of any one of claims 86-93, wherein the conductivity of the target fluidic droplet is less than the conductivity of the first and second ion containment systems.

95. The electroporation system of any one of claims 86-94, wherein the conductivity of the first ion containment system is substantially equal to the conductivity of the second ion containment system.

96. The electroporation system of any one of claims 86-95, wherein the first electrode and the second electrode are in a common substrate.

97. The electroporation system of any one of claims 86-95, wherein the first electrode is in a first substrate and the second electrode is in a second substrate.
